(19) United States
(12) Patent Application Publication
(10) Pub. No.: US 2019/0175887 A1
(43) Pub. Date: Jun. 13, 2019

(54) DILATION INSTRUMENT WITH PROXIMALLY LOCATED FORCE SENSOR

(71) Applicant: Acclarent, Inc., Irvine, CA (US)

(72) Inventor: Ehsan Shameli, Irvine, CA (US)

(21) Appl. No.: 15/840,346

(22) Filed: Dec. 13, 2017

(51) Int. Cl. A61M 29/02; A61M 25/06; A61B 17/24

(52) U.S. Cl. CPC A61M 29/02; A61M 25/0662; A61M 25/09; A61M 2205/332; A61B 17/24

(57) ABSTRACT

An apparatus includes a body, a guide member, an elongate translating member, an actuator, and a force measuring feature. The guide member is coupled to and extends distally from the body, and defines a longitudinal axis. The elongate translating member is operatively coupled with the body and is slidably disposed relative to the guide member. The elongate translating member is configured to translate relative to the body between a retracted position and an extended position for accessing an anatomical passageway. The actuator is coupled to the elongate translating member, and is selectively movable relative to the body to actuate the elongate translating member between the retracted and extended positions. The force measuring feature is operatively coupled with the actuator, and is configured to detect and measure an axial force exerted on the elongate translating member by the actuator when the elongate translating member moves between the retracted and extended positions.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guidewire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Irvine, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein. An example of such an endoscope is the Acclarent Cyclops™ Multi-Angle Endoscope by Acclarent, Inc. of Irvine, Calif.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein. An example of such an illuminating guidewire is the Relieva Luma Sentry™ Sinus Illumination System by Acclarent, Inc. of Irvine, Calif.

While several systems and methods have been made and used in ENT procedures, it is believed that no one prior to the inventors has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1A depicts a perspective view of an exemplary dilation instrument system including a dilation instrument having a guidewire and a dilation catheter, showing the guidewire in a proximal position, and the dilation catheter in a proximal position;

FIG. 1B depicts a perspective view of the dilation instrument system of FIG. 1A, showing the guidewire in a distal position, and the dilation catheter in the proximal position;

FIG. 1C depicts a perspective view of the dilation instrument system of FIG. 1A, showing the guidewire in the distal position, the dilation catheter in a distal position, and a dilator in a non-dilated state;

FIG. 1D depicts a perspective view of the dilation instrument system of FIG. 1A, showing the guidewire in the distal position, the dilation catheter in the distal position, and the dilator in a dilated state;

FIG. 2 depicts a plurality of exemplary variations of a guide catheter of the dilation instrument of FIG. 1A;

FIG. 3 depicts a side elevational view of a distal end portion of the guidewire of the dilation instrument of FIG. 1A;

FIG. 4 depicts a side sectional view of the distal end portion of the guidewire of FIG. 3;

FIG. 5 depicts a side elevational view of the dilation instrument of FIG. 1A, showing the dilation instrument with a force measuring feature according to a first exemplary variation;

FIG. 6 depicts a schematic side elevational view of a force measuring feature according to a second exemplary variation, configured for use with the dilation instrument of FIG. 1A;

FIG. 7 depicts a schematic view showing steps of an exemplary method of monitoring axial force exerted on the guidewire and/or the dilation catheter of the dilation instrument of FIG. 1A during a dilation procedure;

FIG. 8 depicts a schematic view of an exemplary guidewire illumination system incorporating the guidewire of FIGS. 3 and 4;

FIG. 9 depicts an enlarged schematic view of a distal end portion of the guidewire of the illumination system of FIG. 8;

FIG. 10 depicts a schematic view of the dilation instrument of FIG. 1A with the guidewire illumination system of FIG. 8, showing the dilation instrument positioned to dilate the ostium of the maxillary sinus of a patient, and showing the guidewire projecting light through the ostium; and

FIG. 11 depicts a schematic view showing steps of an exemplary method of implementing the guidewire illumination system of FIG. 8 to detect structural deformation of the guidewire during use.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. OVERVIEW OF EXEMPLARY DILATION INSTRUMENT SYSTEM

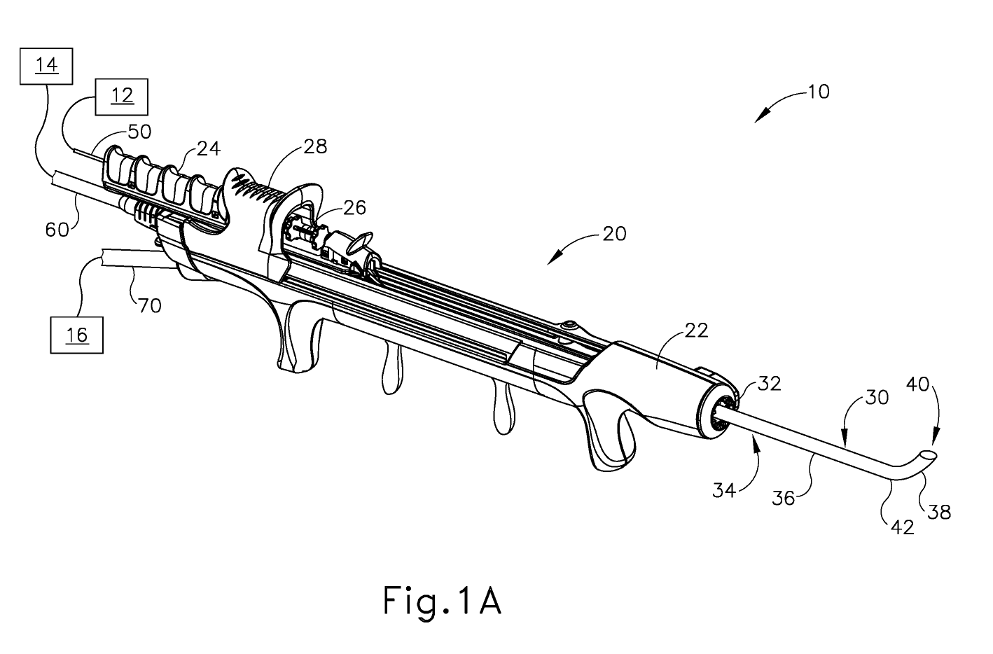

FIGS. 1A-1D show an exemplary dilation instrument system (10) operable to dilate an anatomical passageway of a patient, such as the ostium of a paranasal sinus, a Eustachian tube, or various other anatomical passageways located within the ear, nose, or throat, for example. Dilation instrument system (10) of the present example comprises a guidewire light source (12), an inflation source (14), an irrigation fluid source (16), and a dilation instrument (20).

In the present example, inflation source (14) comprises a source of saline. However, it should be understood that any other suitable source of fluid (liquid or otherwise) may be used. Also in the present example, irrigation fluid source (16) comprises a source of saline. Again, any other suitable source of fluid may be used. It should also be understood that irrigation fluid source (16) may be omitted in some versions.

Dilation instrument (20) of the present example comprises a handle body (22) and movable actuators in the form of a guidewire slider (24), a guidewire spinner (26), and a dilation catheter slider (28) each movably coupled to handle body (22). Handle body (22) is sized and configured to be gripped by a single hand of a human operator. Sliders (24, 28) and spinner (26) are positioned and configured to be manipulated by the same hand that grasps handle body (22). It should therefore be understood that dilation instrument (20) may be fully operated by a single hand of a human operator. As described in greater detail below, dilation instrument (20) further comprises a guide catheter (30), a guidewire (50), and a dilation catheter (60), each operatively coupled with handle body (22).

A. Exemplary Guide Catheter

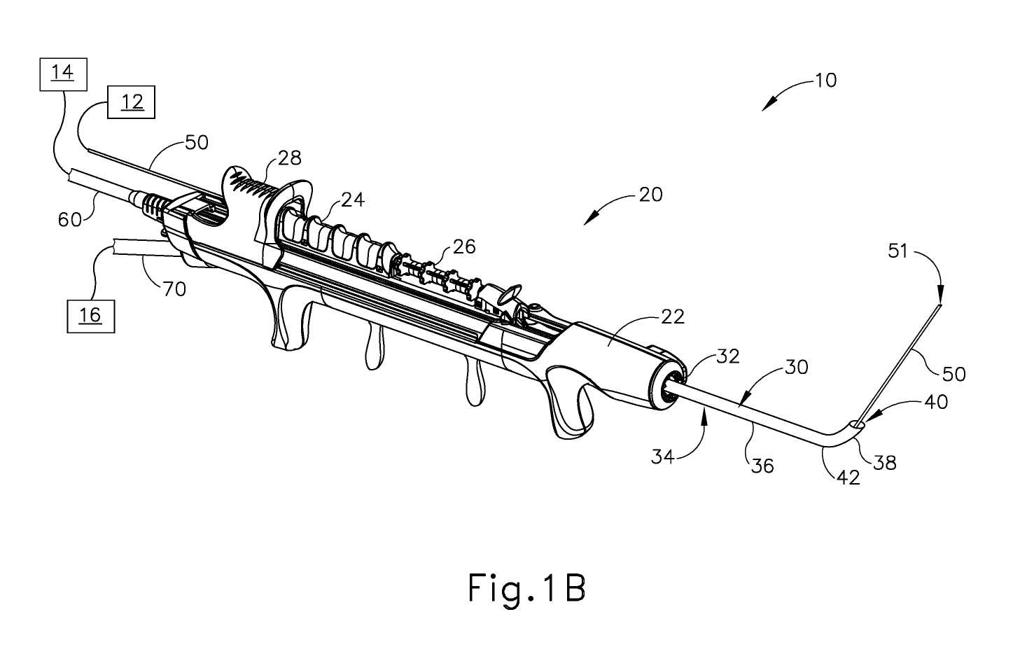

Guide catheter (30) is removably coupled to and extends distally from a distal end of handle body (22). Guide catheter (30) includes a proximal hub (32) (see FIG. 2) and a tubular shaft (34) extending distally from proximal hub (32). Catheter shaft (34) has a straight proximal catheter shaft portion (36), and a bent distal catheter shaft portion (38) terminating at an open distal end (40) defining an atraumatic tip. Distal catheter shaft portion (38) may have a smaller diameter than proximal catheter shaft portion (36). Proximal hub (32) is configured to releasably secure guide catheter (30) to handle body (22), and tubular shaft (34) is configured to slidably receive and guide dilation catheter (60) therethrough, as described below. In some versions, guide catheter (30) may be selectively positionable in a plurality of rotational orientations relative to handle body (22) to facilitate access to various anatomical structures within a patient, such as ostia of various paranasal sinuses.

In the present example, distal catheter shaft portion (38) includes a preformed bend (42) that causes shaft distal end (40) to extend and open along an axis that is angled relative to a longitudinal axis defined by proximal catheter shaft portion (36). Proximal and distal catheter shaft portions (36, 38) may each formed of a substantially rigid material (e.g., rigid metal, and/or rigid plastic, etc.), such that guide catheter (30) maintains a consistent configuration of bend (42) during use of dilation instrument (20). For instance, proximal catheter shaft portion (36) may be formed of a first rigid material, and distal catheter portion (38) may be formed of a second, less rigid material.

As shown in FIG. 2, bend (42) of distal catheter portion (38) may be formed with a variety of suitable bend angles. Each different bend angle may facilitate access to a corresponding paranasal sinus or other anatomical structure(s). For instance, guide catheter (30a) has a preformed bend (42a) defining a bend angle of approximately 0 degrees. Guide catheter (30b) has a preformed bend (42b) defining a bend angle of approximately 30 degrees. Guide catheter (30c) has a preformed bend (42c) defining a bend angle of approximately 70 degrees. Guide catheter (30d) has a preformed bend (42d) defining a bend angle of approximately 90 degrees. Guide catheter (30e) has a preformed bend (42e) defining a bend angle of approximately 110 degrees. It will be understood that these bend angles are merely exemplary, and that guide catheter (30) may be provided with a bend angle of any suitable degree.

By way of example only, guide catheters (30, 30a, 30b, 30c, 30d, 30e) may be constructed and operable in accordance with at least some of the teachings of U.S. Pat. No. 8,894,614, entitled “Devices, Systems, and Methods Useable for Treating Frontal Sinusitis,” issued Nov. 25, 2014, the disclosure of which is incorporated by reference herein; U.S. Pat. No. 7,654,997, entitled “Devices, Systems and Methods for Diagnosing and Treating Sinusitis and Other Disorders of the Ears, Nose, and/or Throat,” issued Feb. 2, 2010, the disclosure of which is incorporated by reference herein; U.S. Pat. No. 7,803,150, entitled “Devices, Systems and Methods Useable for Treating Sinusitis,” issued Sep. 28, 2010, the disclosure of which is incorporated by reference herein; and/or U.S. Pub. No. 2006/0004323, entitled “Apparatus and Methods for Dilating and Modifying Ostia of Paranasal Sinuses and Other Intranasal or Paranasal Structures,” published Jan. 5, 2006, the disclosure of which is incorporated by reference herein.

B. Exemplary Guidewire

Guidewire (50) of dilation instrument (20) is slidably and coaxially disposed within guide catheter (30). Guidewire slider (24) is secured to guidewire (50) such that translation of guidewire slider (24) relative to handle body (22) provides corresponding translation of guidewire (50) relative to handle body (22), through guide catheter (30). In particular, translation of guidewire slider (24) from a proximal position (FIG. 1A) to a distal position (FIG. 1B) causes corresponding translation of guidewire (50) from a proximal position (FIG. 1A) to a distal position (FIG. 1B). When guidewire (50) is in a distal position, a distal end (51) of guidewire (50) protrudes distally from open distal end (40) of guide catheter (30). Guidewire spinner (26) is operable to rotate guidewire (50) about the longitudinal axis of guidewire (50), relative to handle body (22). Guidewire spinner (26) is coupled with guidewire slider (24) such that guidewire spinner (26) translates longitudinally with guidewire slider (24).

In some versions, at least a portion of the length of guidewire (50) (e.g., approximately 7 inches) may be coated in one or more materials, such as silicone, for example. Various other suitable coating materials will be apparent to those of ordinary skill in the art in view of the teachings herein. Additionally, in some versions a distal portion of guidewire (50) may include a preformed bend, for example as disclosed in U.S. Provisional Pat. App. No. 62/453,220, entitled “Navigation Guidewire with Interlocked Coils,” filed Feb. 1, 2017, the disclosure of which is incorporated by reference herein. In such versions, the preformed bend and the rotatability provided via guidewire spinner (26) may facilitate alignment and insertion of guidewire distal end (51) into a sinus ostium, a Eustachian tube, or another anatomical passageway to be dilated.

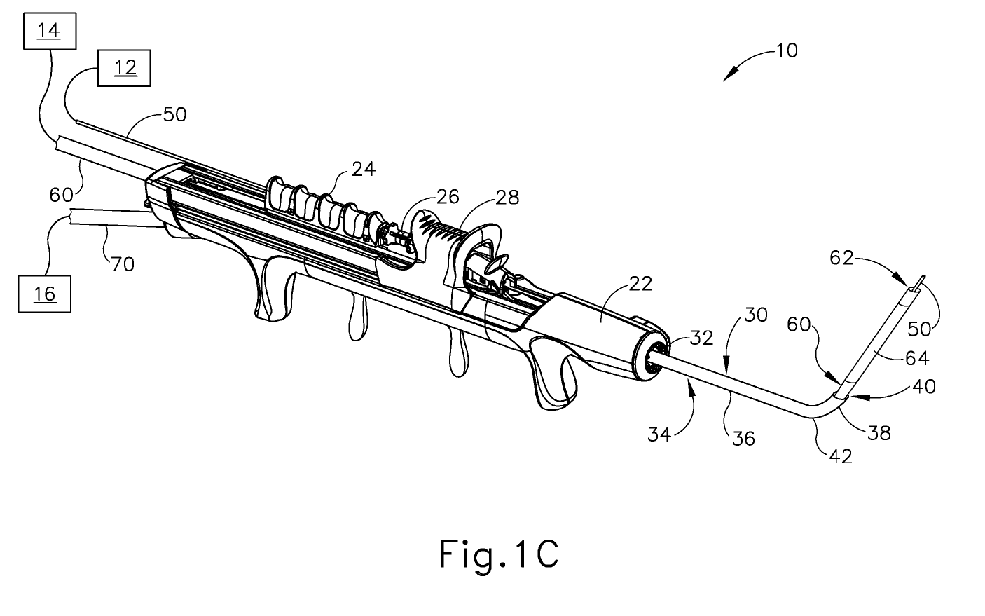

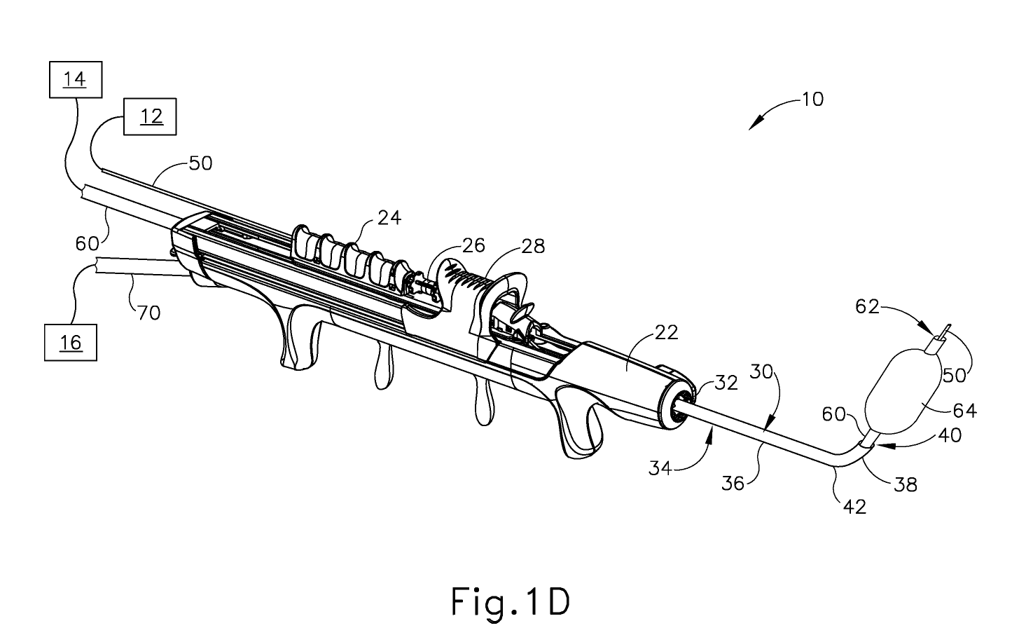

As shown in FIGS. 3 and 4, guidewire (50) of the present example comprises a coil (52) positioned about a core wire (54). An illumination fiber (56) extends along the interior of core wire (54) and terminates distally in an atraumatic lens (58). A connector (59) at the proximal end of guidewire (50) enables optical coupling between illumination fiber (56) and guidewire light source (12). Illumination fiber (56) may comprise one or more optical fibers. Lens (58) is configured to project light when illumination fiber (56) is illuminated by light source (12), such that illumination fiber (56) transmits light from light source (12) to lens (58). As described in greater detail below, illumination fiber (56) and lens (58) may be configured to receive light that is reflected back from anatomical surfaces. The reflected light is then analyzed for monitoring the location of distal end (51) of guidewire (50) relative to the surrounding anatomical structures, and/or for detecting structural deformation of guidewire (50) in real-time during a dilation procedure.

In some examples, the distal end portion of guidewire (50) is more flexible than the proximal end portion of guidewire (50). Additionally, in some examples, guidewire (50) may include indicia along at least part of its length (e.g., the proximal portion) to provide the operator with visual feedback indicating the depth of insertion of guidewire (50) relative to dilation catheter (20). By way of example only, guidewire (50) may be configured in accordance with at least some of the teachings of U.S. Pat. No. 9,155,492, incorporated by reference above. In some versions, guidewire (50) may be configured similar to the Relieva Luma Sentry™ Sinus Illumination System by Acclarent, Inc. of Irvine, Calif. Other suitable forms that guidewire (50) may take will be apparent to those of ordinary skill in the art in view of the teachings herein.

C. Exemplary Dilation Catheter

Dilation catheter (60) of dilation instrument (20) is slidably and coaxially disposed within guide catheter (30), and over guidewire (50). Dilation catheter slider (28) is secured to dilation catheter (60) such that translation of dilation catheter slider (28) relative to handle body (22) provides corresponding translation of dilation catheter (60) relative to handle body (22). In particular, translation of dilation catheter slider (28) from a proximal position (FIG. 1B) to a distal position (FIG. 1C) causes corresponding translation of dilation catheter (60) from a proximal position (FIG. 1B) to a distal position (FIG. 1C). When dilation catheter (60) is in a distal position, a distal portion of dilation catheter (60) protrudes distally from open distal end (64) of guide catheter (30). As can also be seen in FIG. 1C, a distal portion of guidewire (50) protrudes distally from an open distal end (62) of dilation catheter (60) when guidewire (50) and dilation catheter (60) are both in distal positions.

Dilation catheter (60) of the present example includes a dilation member in the form of a non-extensible balloon (64) located just proximal to open distal end (62) of dilation catheter (60). Balloon (64) is in fluid communication with inflation source (14). Inflation source (14) is configured to communicate fluid (e.g., saline, etc.) to and from balloon (64) to thereby transition balloon (64) between a non-inflated state and an inflated state. FIG. 1C shows balloon (64) in a non-inflated state. FIG. 1D shows balloon (64) in an inflated state. Though not shown, it should be understood that dilation catheter (60) may include at least two, separate lumens that are fluidically isolated from one another. The first lumen may provide a pathway for fluid communication between balloon (64) and inflation source (14), while the second lumen provides a pathway to slidably receive guidewire (50).

In some versions, inflation source (14) comprises a manually actuated source of pressurized fluid. In some such versions, the manually actuated source of pressurized fluid may be configured and operable in accordance with at least some of the teachings of U.S. Pub. No. 2014/0074141, entitled “Inflator for Dilation of Anatomical Passageway,” published Mar. 13, 2014, the disclosure of which is incorporated by reference herein. Other suitable configurations that may be used to provide a source of pressurized fluid will be apparent to those of ordinary skill in the art in view of the teachings herein.

While dilation catheter (60) of the present example is configured to transition between a non-dilated state (FIG. 1C) and a dilated state (FIG. 1D) based on the communication of fluid to and from balloon (64), it should be understood that dilation catheter (60) may include various other kinds of structures to serve as a dilator. By way of example only, balloon (64) may be replaced with a mechanical dilator. Dilation catheter (60) may be constructed and operable in accordance with any of the various references cited herein. In some versions, dilator catheter (60) is configured and operable similar to the Relieva Ultirra™ Sinus Balloon Catheter by Acclarent, Inc. of Irvine, Calif. In some other versions, dilator catheter (60) is configured and operable similar to the Relieva Solo Pro™ Sinus Balloon Catheter by Acclarent, Inc. of Irvine, Calif. Other suitable variations of dilation catheter (60) will be apparent to those of ordinary skill in the art in view of the teachings herein.

In the present example, guidewire (50) is coaxially disposed within dilation catheter (60), which is coaxially disposed within guide catheter (30). In some other versions, guidewire (50) is omitted and dilation catheter (60) is slidably disposed about the exterior of an internal malleable guide member. In some other versions, guidewire (50) is slidably disposed about the exterior of the internal malleable guide member, and dilation catheter (60) is slidably disposed about the exterior of guidewire (50). In still other versions, guidewire (50) is slidably disposed within the interior of the malleable guide member, and dilation catheter (60) is slidably disposed about the exterior of the malleable guide member.

By way of example only, versions of dilation instrument (20) that include a malleable guide member may be constructed and operable in accordance with at least some of the teachings of U.S. Pub. No. 2016/0310714, entitled “Balloon Dilation System with Malleable Internal Guide,” published Oct. 27, 2016, the disclosure of which is incorporated by reference herein. As another merely illustrative example, versions of dilation instrument (20) that include a malleable guide member may be constructed and operable in accordance with at least some of the teachings of U.S. Pub. No. 2017/0120020, entitled “Apparatus for Bending Malleable Guide of Surgical Instrument,” published May 4, 2017, the disclosure of which is incorporated by reference herein; and/or U.S. Pub. No. 2012/0071857, entitled “Methods and Apparatus for Treating Disorders of the Sinuses,” published Mar. 22, 2012, the disclosure of which is incorporated by reference herein.

D. Exemplary Irrigation Features

In some instances, it may be desirable to irrigate an anatomical site. For instance, it may be desirable to irrigate a paranasal sinus and nasal cavity after dilation catheter (60) has been used to dilate an ostium or other drainage passageway associated with the paranasal sinus. Such irrigation may be performed to flush out blood, etc. that may be present after a dilation procedure. In some such cases, guide catheter (30) may be allowed to remain in the patient while guidewire (50) and dilation catheter (60) are removed. A dedicated irrigation catheter (not shown) may then be inserted into guide catheter (30) and coupled with irrigation fluid source (16) via irrigation tube (70), to enable irrigation of the anatomical site in the patient.

An example of an irrigation catheter that may be fed through guide catheter (30) to reach the irrigation site after removal of dilation catheter (30) is the Relieva Vortex® Sinus Irrigation Catheter by Acclarent, Inc. of Irvine, Calif. Another example of an irrigation catheter that may be fed through guide catheter (30) to reach the irrigation site after removal of dilation catheter (60) is the Relieva Ultirra® Sinus Irrigation Catheter by Acclarent, Inc. of Irvine, Calif. In some other versions, dilation catheter (60) may include an additional irrigation lumen and an associated set of irrigation ports located near distal end (62), such that dilation catheter (60) may be coupled with irrigation fluid source (16) via tube (70). Thus, a separate, dedicated irrigation catheter is not necessarily required in order to provide irrigation.

By way of example only, irrigation may be carried out in accordance with at least some of the teachings of U.S. Pat. No. 7,630,676, entitled “Methods, Devices and Systems for Treatment and/or Diagnosis of Disorders of the Ear, Nose and Throat,” issued Dec. 8, 2009, the disclosure of which is incorporated by reference herein; and U.S. Pat. No. 9,095,646, entitled “Devices and Methods for Transnasal Dilation and Irrigation of the Sinuses,” issued Aug. 4, 2015, the disclosure of which is incorporated by reference herein. Of course, irrigation may be provided in the absence of a dilation procedure; and a dilation procedure may be completed without also including irrigation. It should therefore be understood that irrigation fluid source (16) and tube (70) are optional components.

In various other examples, dilation instrument system (10) may include additional or alternative features in accordance with at least some of the teachings of U.S. Pat. No. 8,777,926, entitled “Apparatus and Methods for Dilating and Modifying Ostia of Paranasal Sinuses and Other Intranasal or Paranasal Structures,” issued Jul. 15, 2014, the disclosure of which is incorporated by reference herein.

E. Exemplary Use of Dilation Instrument

Dilation instrument (10) may be used to dilate various types of anatomical passageways of a patient, such as the ostium of a paranasal sinus, a Eustachian tube, or various other anatomical passageways located within the patient's ear, nose, or throat, for instance. In an exemplary procedure for dilating an ostium of a paranasal sinus, an operator manipulates instrument (10) to insert distal end (40) of guide catheter (30) through a nose opening and into the nasal cavity. The operator advances guide catheter (30) further to position distal end (40) adjacent to the opening of the targeted ostium to be dilated. Once distal end (40) is suitably positioned relative to the ostium, the operator actuates guidewire slider (24) distally relative to handle body (22) to thereby advance guidewire (50) distally into the ostium. The operator may rotate guidewire spinner (26) as needed to rotate distal end (51) of guidewire (50) relative to the ostium.

Once distal end (51) of guidewire (50) is suitably positioned relative to the ostium, the operator actuates dilation catheter slider (28) distally to thereby advance dilation catheter (60), in a non-dilated state (see, e.g., FIG. 1C), distally over top of guidewire (50) and into the ostium. Once balloon (64) is suitably positioned within the ostium, the operator causes inflation fluid to be directed from fluid source (14) to balloon (64). Balloon (64) expands to its dilated state (see, e.g., FIG. 1D), and thereby dilates the ostium. Balloon (64) is then returned to its non-dilated state (FIG. 1C), and the operator actuates dilation catheter slider (28) proximally to thereby retract dilation catheter (60) proximally from the ostium, into guide catheter (30). The operator then actuates guidewire slider (24) proximally to thereby retract guidewire (50) proximally from the ostium, into guide catheter (30). Optionally, before or after the proximal retraction of guidewire (50), the operator may activate irrigation fluid source (16) to irrigate the sinus to which the dilated ostium opens, as well as the nasal cavity. Finally, following all dilation and irrigation steps, the operator manipulates instrument (10) to withdraw guide catheter (30) from the patient's nose.

As described in greater detail below, dilation procedures using dilation instrument (20) may be enhanced by implementing one or more force measurement features to avoid exertion of excessive axial force, and resulting damage, by the operator on guidewire (50) and/or dilation catheter (60). Additionally, or in the alternative, light transmission features of dilation instrument (20) may be employed to monitor the location of distal end (51) of guidewire (50) relative to surrounding anatomical structures, and/or for detecting structural deformation of guidewire (50) during a procedure.

### II. EXEMPLARY FORCE MEASUREMENT FEATURES OF DILATION INSTRUMENT

During procedures in which an anatomical passageway is dilated using a dilation instrument, such as dilation instrument (20) described above, exertion of excessive axial force on the guidewire during its deployment can undesirably result in plastic deformation of the guidewire in the form of buckling (or “kinking”). For instance, when attempting to overcome resistance encountered by the guidewire as it advances through internal anatomy of the patient, the operator may inadvertently exert a distally directed axial force on the guidewire, via an actuator, that overcomes the column strength of the guidewire and causes kinking. Such kinking is generally undesirable, and can prevent the guidewire from retracting fully back into the dilation instrument.

The dilation catheter of a dilation instrument, such as instrument (20), may also be subject to inadvertent kinking during deployment within a patient, for example under similar circumstances to those described above. Additionally, if an operator attempts to withdraw the dilation catheter proximally from an anatomical passageway before the dilation member is sufficiently deflated, or otherwise retracted, the dilation member can cause undesirable trauma to internal anatomy. In that regard, a partially inflated balloon presents a larger outer diameter than a deflated balloon. Exerting excessive axial force on the dilation catheter to thereby withdraw the dilation catheter from the anatomical passageway while the balloon remains partially inflated can result in the balloon engaging and causing trauma to internal anatomy, and also possibly rupturing the balloon.

The exemplary force measuring features described below enable an operator of dilation instrument (20) to monitor the amount of axial force being exerted on guidewire (50) and dilation catheter (60) during distal advancement and proximal retraction, and thereby enable the operator to ensure that excessive axial force is not exerted and avoid the undesirable results described above.

A. Exemplary Force Sensor

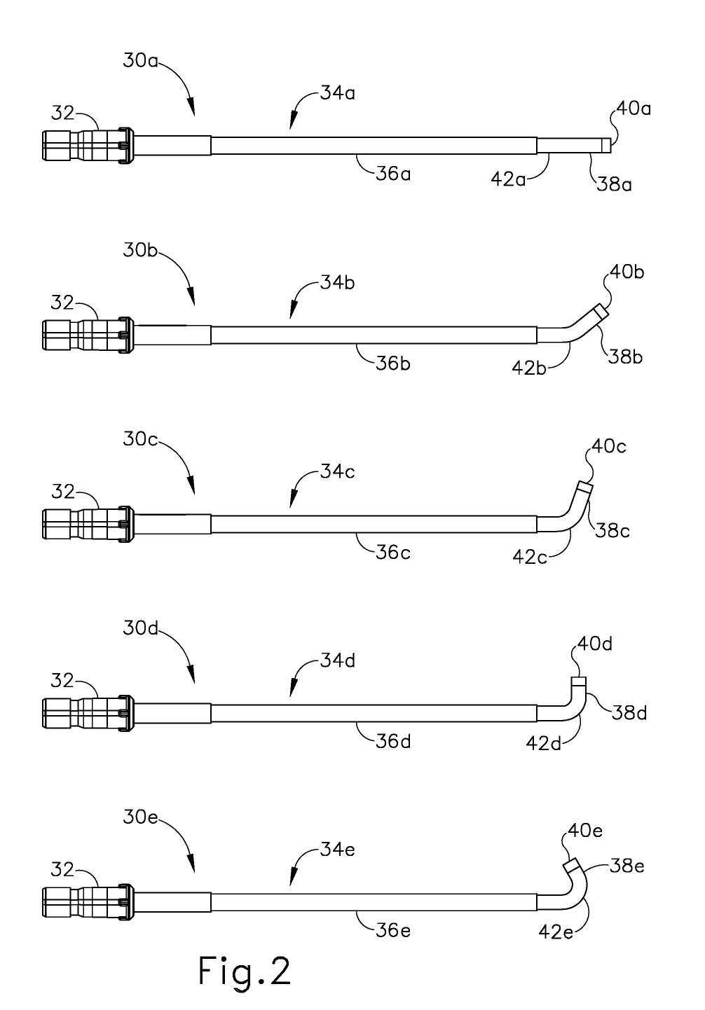

FIG. 5 shows an exemplary configuration of dilation instrument (20) in which instrument (20) includes a force measuring feature in the form of an electronic force sensor (100) in communication with a controller (102). Force sensor (100) is configured to detect and measure the amount of axial force exerted by a user on either or both of guidewire (50) and dilation catheter (60) during their distal advancement and proximal retraction, and communicate the measured axial force to controller (102) in real time during use. Force sensor (100) may be in the form of a transducer, such as a load cell of the type made available by FUTEK Advanced Sensor Technology, Inc. of Irvine, Calif., for example.

As shown in FIG. 5, force sensor (100) is operatively coupled to guidewire slider (24) and dilation catheter slider (28), such that an axial force exerted on either of sliders (24, 28) is transmitted to force sensor (100). Accordingly, when an operator exerts an axial force on either of sliders (24, 28) to thereby actuate guidewire (50) or dilation catheter (60) proximally or distally, force sensor (100) detects the axial force and generates a signal corresponding to the detected axial force. Force sensor (100) transmits the force signal to controller (102), which analyzes the signal and may take various actions in response. In one example, controller (102) may instruct that the magnitude of the measured force be displayed to the operator, for example via a visual display (not shown). In another example, controller (102) may compare the measured force magnitude to a predetermined force magnitude, and may provide an indication to the operator if the measured force magnitude is less than, greater than, or equal to the predetermined force magnitude. Such an indication may be provided in the form of a visual, audible, and/or tactile indication by one or more suitable mechanisms that will be readily apparent to those of ordinary skill in the art.

Controller (102) may be provided internally or externally of dilation instrument (20). For instance, in one example controller (102) may be a component of a data acquisition system (not shown) arranged externally of but in communication with dilation instrument (20). The data acquisition system may include a visual display and one or more user interface devices, for example. In other versions, controller (102) may be arranged within a portion of dilation instrument (20), such as handle body (22). In such versions, the one or more indication mechanisms described above may be incorporated into the structure of dilation instrument (20) as well, for example.

While only one force sensor (100) is shown in the present example, it will be appreciated that two or more force sensors (100) may be provided, and may be arranged at various suitable locations on dilation instrument (20). For instance, a first force sensor (100) may be coupled to guidewire slider (24) or another suitable component coupled to guidewire (50). Similarly, a second force sensor (100) may be coupled to dilation catheter slider (28) or another suitable component coupled to dilation catheter (30). The two or more force sensors (100) may each be in communication with controller (102) and be configured to provide respective force signals to controller (102).

B. Exemplary Mechanical Force Indicator Mechanism

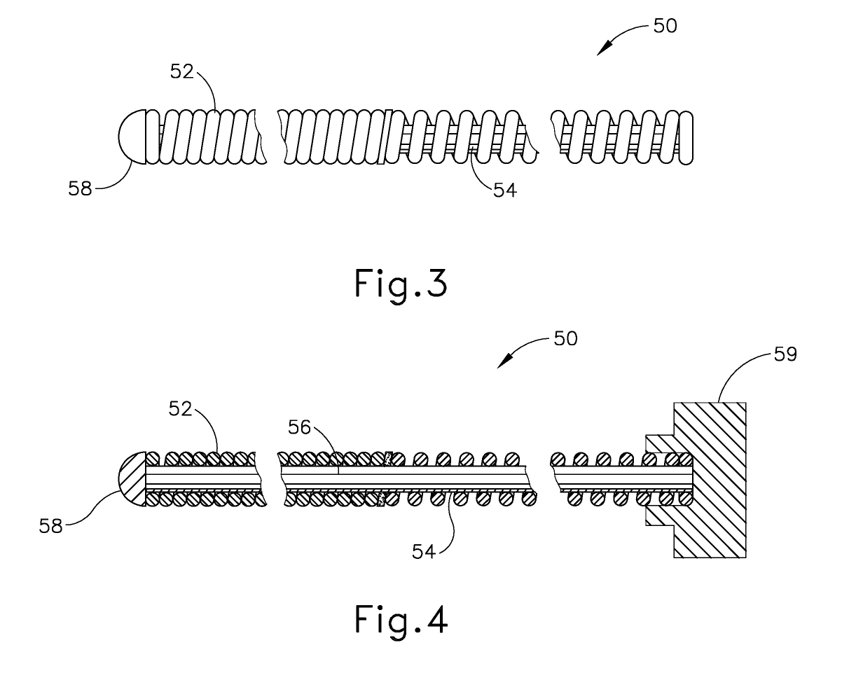

FIG. 6 shows another exemplary configuration of dilation instrument (20) in which instrument (20) includes a force measuring feature in the form of a mechanical force indicator mechanism (110). Mechanical force indicator mechanism (110) is functionally similar to force sensor (100) in that indicator mechanism (110) is configured to detect and measure the amount of axial force exerted by an operator on guidewire (50) or dilation catheter (60) during their distal advancement and proximal retraction. Whereas force sensor (100) communicates the measured axial force to a controller (102), which then dictates if and how the force measurement is communicated to the operator, indicator mechanism (110) communicates the measured axial force directly to the operator during use, as described below.

As indicated by reference numerals in FIG. 6, mechanical force indicator mechanism (110) may be coupled to either of guidewire (50) or dilation catheter (60) for measuring an axial force exerted by an operator on the respective structure. While only one indicator mechanism (110) is shown, it will be appreciated that multiple indicator mechanisms (110) may be provided on the same dilation instrument (20). For instance, a first indicator mechanism (110) may be coupled to guidewire slider (24), or another suitable component coupled to guidewire (50), and be configured to measure and indicate axial force exerted by the operator on guidewire (50) during use. Similarly, a second indicator mechanism (110) may be coupled to dilation catheter slider (28), or another suitable component coupled to dilation catheter (60), and be configured to measure and indicate axial force exerted by the operator on dilation catheter (60) during use. Alternatively, a single indicator mechanism (110) may be provided that is configured to measure and indicate axial forces exerted by the operator on guidewire (50) and dilation catheter (60).

Mechanical force indicator mechanism (110) of the present example includes a resilient member in the form of a compression spring (112). In other versions, various other suitable types of resilient members may be employed. As shown in FIG. 6, spring (112) encircles and is coupled to guidewire (50) or dilation catheter (60) with one or more constraining elements (114), which enable spring (112) to compress relative to the guidewire (50) or dilation catheter (60) while remaining within a fixed longitudinal region of guidewire (50) or dilation catheter (60). Accordingly, in use spring (112) translates proximally and distally relative to handle body (22) with guidewire (50) or dilation catheter (60), while maintaining the ability to compress axially relative to guidewire (50) or dilation catheter (60), as described in greater detail below. In the present example, constraining elements (114) are shown positioned at the proximal and distal ends of spring (112), though it will be appreciated that constraining elements (114) may be positioned at various other suitable locations in other examples, such as at a medial portion of spring (112).

A proximal end of spring (112) is coupled to (e.g., bounded proximally by) a proximal end of slider (24, 28), and a distal end of spring (112) is coupled to (e.g., bounded distally by) a distal end of slider (24, 28). Accordingly, as an operator exerts an axial force on slider (24, 28), slider (24, 28) transmits the axial force to spring (112), which then compresses axially by some amount and transmits the axial force to guidewire (50) or dilation catheter (60).

Mechanical force indicator mechanism (110) further includes a measurement scale (116) having a plurality of visible indicia markings (118) corresponding to axial force magnitude. An indicator element (120) is rigidly coupled to the shaft portion of guidewire (50) or dilation catheter (60), and is configured to align with markings (118) of measurement scale (116) to indicate to the operator a magnitude of axial force being exerted on guidewire (50) or dilation catheter (60) during proximal advancement and distal retraction. The components of indicator mechanism (110) may be configured such that when zero axial force is exerted on slider (24, 28), spring (112) does not compress and indicator element (120) interacts with indicia markings (118) to indicate an axial force magnitude of zero, as shown in FIG. 6. In the present example, this zero-position of indicator element (120) is arranged in the approximate middle of measurement scale (116). Accordingly, exertion of a distally directed force on slider (24, 28) (e.g., during device advancement) results in indicator element (120) registering a force magnitude on a proximal portion of measurement scale (116). In contrast, exertion of a proximally directed force on slider (24, 28) (e.g., during device retraction) results in indicator element (120) registering a force magnitude on a distal portion of measurement scale (116). It will be appreciated that measurement scale (116), indicia markings (118), and indicator element (120) may be configured in various other manners in other versions.

Those of ordinary skill in the art will readily appreciate that axial deflection of spring (112) is directly proportional to the axial force (or “load”) exerted on spring (112). This relationship, along with a known spring constant of spring (112), may be implemented to suitably space indicia markings (118) on measurement scale (116) relative to the zero-position of indicator element (120), such that each marking (118) represents a specific axial deflection and corresponding axial force magnitude exerted on spring (112) and thus on guidewire (50) or dilation catheter (60). Further, any suitable quantity and denomination of markings (118) may be provided. Additionally, indicia markings (118) may comprise one or more numerals, letters, symbols, shapes, colors, or other visual indicia suitable to assist an operator in identifying an axial force magnitude being indicated by force indicator mechanism (110) during use.

### C. Exemplary Method of Monitoring Axial Force with Force Measurement Feature

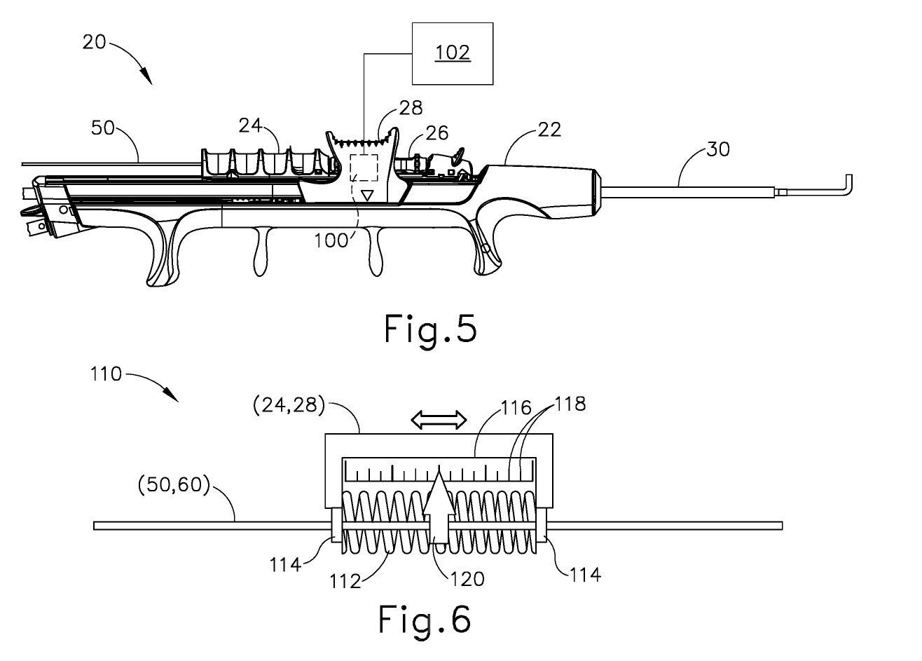

FIG. 7 is a schematic view showing steps of an exemplary method (130) of monitoring axial force exerted on guidewire (50) or the dilation catheter (60) of the dilation instrument (20) during a surgical procedure using either of the force measuring features (100, 110) described above. In step (132), the operator exerts an axial force on guidewire (50) or dilation catheter (60), via sliders (24, 28), to thereby actuate guidewire (50) or dilation catheter (60) relative to handle body (22). As described above, this axial force may be exerted in a distal direction to advance the guidewire (50) or dilation catheter (60) through an anatomical passageway toward an extended position, or in a proximal direction to retract the guidewire (50) or dilation catheter (60).

In step (134), force measuring feature (100, 110) detects and measures the axial force exerted on guidewire (50) or dilation catheter (60) by the operator. In step (136), this measured force (F_{M}) is compared to a predetermined threshold force (F_{T}), which may correspond to a column strength of guidewire (50) or dilation catheter (60) with a suitable factor of safety, for instance. In examples in which the force measuring feature is in the form of force sensor (100), this determination may be made by controller (102) or by the operator based on an indication (e.g., a visual display) provided to the operator. In examples in which the force measuring feature is in the form of mechanical force indicator mechanism (110), this determination is made by the operator based on an indication provided by indicator element (120) and measurement scale (116) of indicator mechanism (110).

In the present example, if the measured force (F_{M}) is less than or equal to the predetermined threshold force (F_{T}), the operator proceeds to step (138) in which the operator continues exerting the axial force on guidewire (50) or dilation catheter (60), via sliders (24, 28). Throughout this continued exertion of axial force, the assessment of step (136) is repeated. Once it is determined that the measured force (F_{M}) exceeds the predetermined threshold force (F_{T}), the operator ceases the exertion of axial force, as represented by step (140). In examples in which the force measuring feature is in the form of force sensor (100), controller (102) may provide an indication to the operator that this condition has been reached, for example with a visual, audible, and/or tactile indication as described above.

Dilation instrument (20) is configured in the present example such that even when force measuring feature (100, 110) detects and indicates to the operator that the measured force (F_{M}) exceeds the predetermined threshold force (F_{T}), guidewire (50) and dilation catheter (60) remain actuatable relative to handle body (22) of dilation instrument (20). In that regard, it will be appreciated that force measuring features (100, 110) function merely to provide an indication to the operator of the current axial force exerted on guidewire (50) or dilation catheter (60). In other examples, however, dilation instrument (20) may include one or more mechanisms (not shown) operable to restrict continued actuation of guidewire (50) and/or dilation catheter (60) relative to handle body (22) when the predetermined threshold force (F_{T}) is exceeded.

### III. EXEMPLARY GUIDEWIRE ILLUMINATION SYSTEM

A. Overview of Guidewire Illumination System

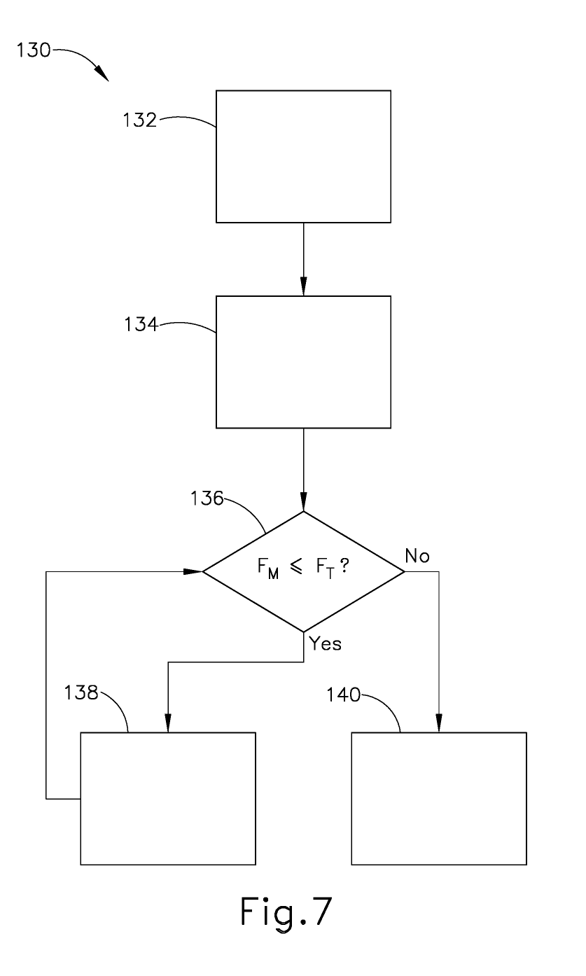

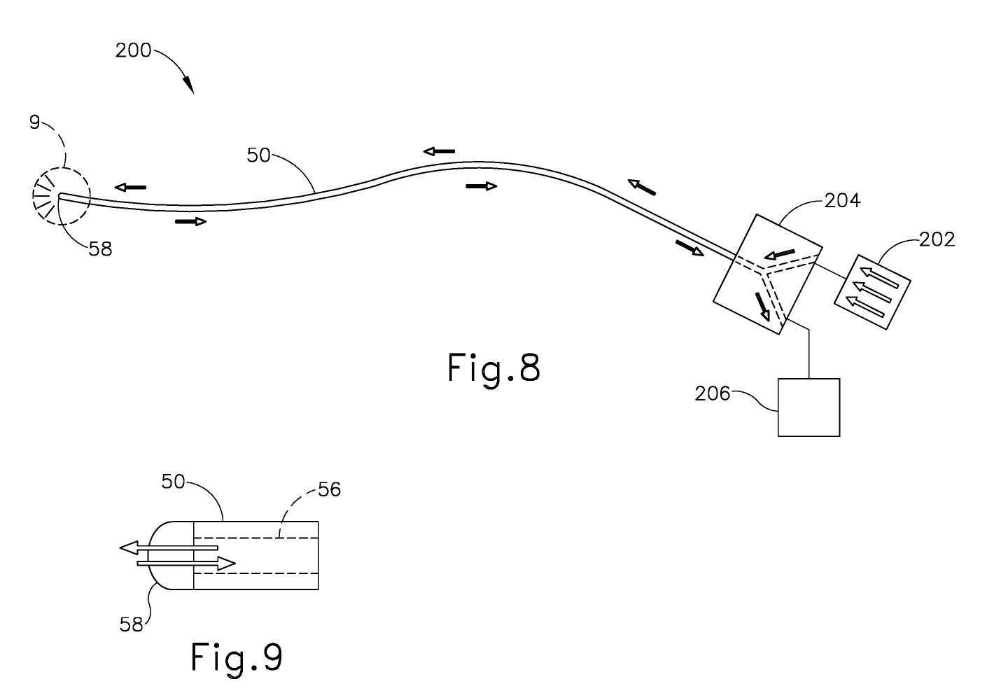

FIGS. 8 and 9 show an exemplary guidewire illumination system (200) comprising guidewire (50) of dilation instrument (20) described above, a conventional light source (202), a conventional beam splitter (204), and a conventional light detector (206). Light source (202) may comprise any suitable type of light source, and may include various components, including but not limited to a laser, a beam collimator, focusing optics, etc. Light source (202) may be operable to communicate any suitable kind of light, including but not limited to white/visible light, near-infrared light, infrared light, etc. As shown in FIG. 8, beam splitter (204) is operable to transmit light distally from light source (202) to illumination fiber (56) of guidewire (50), for distal emission through lens (58). Beam splitter (204) is further operable to transmit reflected light, received from lens (58) via illumination fiber (56), proximally to light detector (206). Light detector (206) includes a sensor that is operable to generate electrical signals in response to reflected light received from beam splitter (204), as described in greater detail below.

Light detector (206), and/or one or more components that are coupled with light detector (206), may further include hardware that is configured to process such electrical signals and generate an output that provides feedback to the operator relating to the light received by light detector (206). Such feedback may include audible feedback (e.g., an audible tone, a voice providing spoken words, etc.), visual feedback (e.g., a selectively illuminating LED, a graphical interface providing graphic and/or textual feedback, etc.), and/or tactile feedback (e.g., a feature providing a vibration through a handpiece associated with guide catheter (30), etc.). Various suitable forms that light source (202), beam splitter (204), and light detector (206) (and associated components) may take will be apparent to those of ordinary skill in the art in view of the teachings herein. Similarly, various suitable forms that operator feedback may take will be apparent to those of ordinary skill in the art in view of the teachings herein. In some instances, it may be desirable to configure light detector (206) such that detector (206) is operable to “subtract” any unwanted light from light scattering, reflection, or other optical phenomena so as to improve upon the information indicated by detector (206). Various suitable ways in which such subtraction may be provided will be apparent to those of ordinary skill in the art in view of the teachings herein.

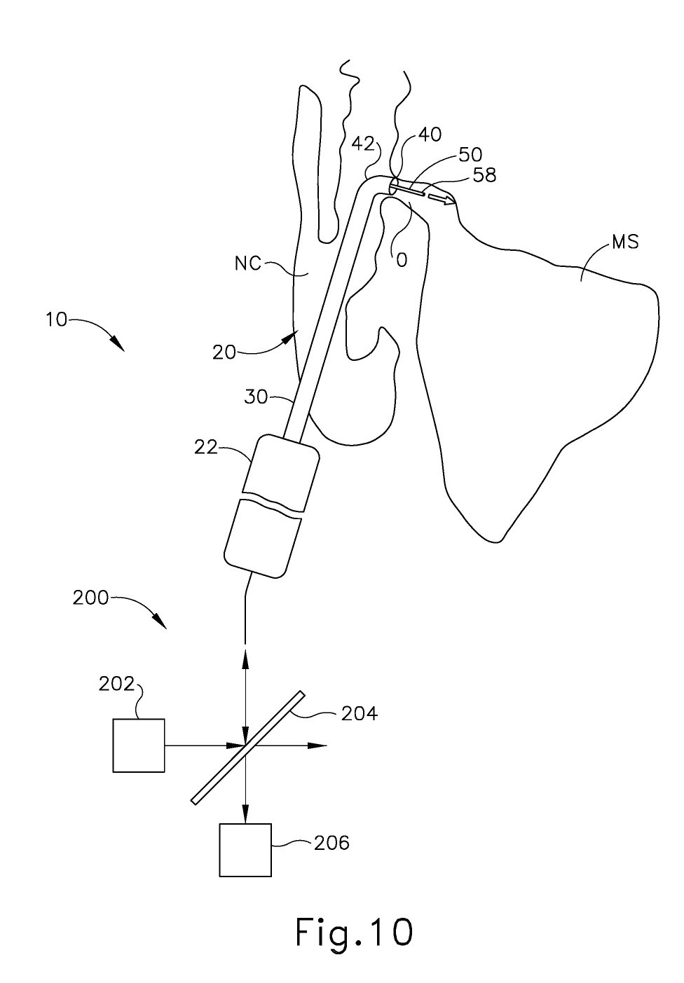

FIG. 10 shows an exemplary dilation procedure that implements dilation instrument system (10) described above to dilate a sinus ostium (O) of a maxillary sinus (MS) of a patient. Dilation instrument system (10) of the present example incorporates guidewire illumination system (200), described above, such that light source (202) comprises light source (12). While the present example is provided in the context of dilating a sinus ostium (O) of maxillary sinus (MS), it should be understood that dilation instrument system (10) may be used in various other procedures. By way of example only, dilation instrument system (10) and variations thereof may be used to dilate a Eustachian tube, a larynx, a choana, a sphenoid sinus ostium, one or more openings associated with one or more ethmoid sinus air cells, the frontal recess, and/or other passageways associated with paranasal sinuses. Moreover, other suitable ways in which dilation instrument system (10) may be used will be apparent to those of ordinary skill in the art in view of the teachings herein.

As shown in FIG. 10, guide catheter (30) is inserted transnasally and advanced through the nasal cavity (NC) to a position within or near the targeted sinus ostium (O). At this stage, inflatable dilator (22) and the distal end of guidewire (50) may be positioned within or proximal to bent distal portion (38) of guide catheter (30). This positioning of guide catheter (30) may be verified endoscopically with an endoscope, by direct visualization, by radiography, and/or by any other suitable method.

After guide catheter (30) has been suitably positioned, the operator may advance guidewire (50) distally through guide catheter (30) such that a distal portion of guidewire (50) passes through the ostium (O) and is oriented toward the cavity of maxillary sinus (MS), as shown in FIG. 10. Simultaneously, guidewire illumination system (200) is activated so that light source (202) projects light toward beam splitter (204), which redirects a portion of the light to transmit distally through illumination fiber (56) of guidewire (50). This transmitted light travels distally through illumination fiber (56) and is emitted from the distal end of guidewire (50) via lens (58), as shown in FIGS. 8 and 9, described above. In some instances, this emitted light may have an intensity sufficient to provide transcutaneous illumination (or “transillumination”) through the patient's face, thereby enabling the operator to visually confirm positioning of the distal end of guidewire (50) within the patient.

At least a portion of the light emitted by guidewire lens (58) may be reflected back by anatomical structures surrounding the distal end of guidewire (50) in a direction toward lens (58), as shown in FIGS. 8 and 9. This reflected light reenters illumination fiber (56) through lens (58), and is transmitted proximally through illumination fiber (56), through beam splitter (204), and to detector (206). Light detector (206) is operable to then determine the presence and characteristics of the reflected light (e.g., intensity, wavelength, etc.), to thereby determine the presence of anatomical structure(s) that are distal to the distal end of guidewire (50). As noted above, based on the detection of light reflected back from such anatomical structure(s), detector (206) and/or components coupled with detector (206) may further provide real-time feedback to the operator concerning the position of guidewire (50) relative to the anatomical structure(s).

For instance, based upon one or more characteristics of the reflected light (e.g., intensity, wavelength, etc.), light detector (206) may determine a distance between the distal end of guidewire (50) and the anatomical structure(s) that surrounds the distal end of guidewire (50), as well as the color of such anatomical structure(s). In addition, detector (206), based upon quantitative optical spectroscopy, optical coherence tomography, and/or other optical processing techniques, may determine and indicate a distance between the distal end of guidewire (50) and the anatomical structure(s), as well as the type and/or pathology of the anatomical structure(s). For instance, as the distal end of guidewire (50) advances toward a wall of the maxillary sinus (MS), as shown in FIG. 10, the intensity of light reflected toward the distal end of guidewire (50) increases. This increase in intensity of reflected light generally indicates that the distal end of guidewire (50) is approaching the wall. As noted above, detector (206) and/or components that are coupled with detector (206) may be configured to provide visual, audible, and/or tactile feedback to an operator based on such information. Guidewire illumination system (200) may be further configured and operable in accordance with one or more teachings of U.S. Pub. No. 2016/0287083, entitled “Illuminating Guidewire With Optical Sensing,” published Oct. 6, 2016, the disclosure of which is incorporated by reference herein.

Once the operator has determined that guidewire (50) is suitably positioned based on optical feedback provided through the reflected light, the operator may advance dilation catheter (20) along guidewire (50) to position dilator (22) in the ostium (O) of the maxillary sinus (MS). The operator may then inflate dilator (22) as described above to dilate the ostium (O). Alternatively, the operator may perform any other desired actions within the maxillary sinus (MS), within the ostium (O), and/or elsewhere.

B. Exemplary Detection of Structural Deformation of Guidewire

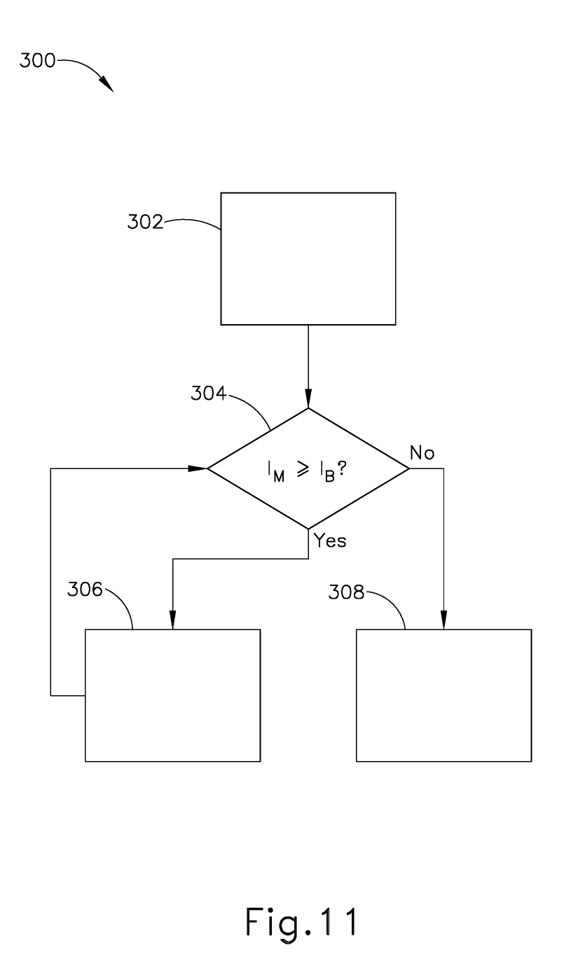

In addition to analyzing characteristics of reflected light to monitor distance between the distal end of guidewire (50) and adjacent anatomical structures, guidewire illumination system (200) may be further configured to analyze characteristics of the reflected light to detect plastic deformation of guidewire (50) in the form of buckling (or “kinking”). FIG. 11 shows steps of an exemplary method (300) for performing this procedure.

In step (302) of method (300), the operator advances guidewire (50) distally through internal anatomy of the patient such that light is emitted distally through guidewire lens (58), and such that at least a portion of the emitted light reflects back into lens (58) and travels proximally through illumination fiber (56) to light detector (206). In subsequent step (304), light detector (206) measures an intensity (I_{M}) of the reflected light, and compares this measured intensity (I_{M}) to a known baseline light intensity (I_{B}). In the present example, this comparison step (304) is performed by a controller integrated within light detector (206). In other examples, the controller may be provided separately from but in communication with light detector (206). The baseline intensity (I_{B}) corresponds to an intensity of light reflected proximally through illumination fiber (56) when guidewire (50) and illumination fiber (56) are in a non-buckled state (i.e., free of kinks). Accordingly, a measured intensity (I_{M}) less than the baseline intensity (I_{B}) is indicative of plastic deformation (e.g., kinking) in the guidewire (50), or of an elastically deformed state of guidewire (50) that is approaching plastic deformation.

If light detector (206) detects that the measured intensity (I_{M}) of the reflected light is greater than or equal to the baseline intensity (I_{B}), detector (206) determines that guidewire (50) remains in a kink-free state. Optionally, light detector (206) may provide an indication of this kink-free state to the operator. As long as guidewire (50) remains kink-free, the operator continues advancing guidewire (50) through the patient, as represented by step (306). Light detector (206) continuously repeats its light intensity analysis performed in step (304) throughout the ongoing advancement of guidewire (50). If and when light detector (206) detects in step (304) that the measured intensity (I_{M}) of the reflected light is less than the baseline intensity (I_{B}), light detector (206) determines that guidewire (50) has reached, or is approaching, a plastically deformed state, for example due to application of excessive axial force on guidewire (50) as generally described above. As indicated by step (308), upon making this determination detector (206) provides an indication of such to the operator. Such an indication may be in the form of a visual, audible, or tactile indication, for example, provided by any suitable indicator mechanism. In response to receiving this indication, the operator may choose to lessen or cease the axial force being exerted on guidewire (50), to thereby avoid initial or further buckling (or “kinking”) of guidewire (50).

### IV. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a body; (b) a guide member coupled to and extending distally from the body, wherein the guide member defines a longitudinal axis; (c) an elongate translating member operatively coupled with the body and slidably disposed relative to the guide member, wherein the elongate translating member is configured to translate relative to the body between a retracted position and an extended position for accessing an anatomical passageway; (d) an actuator coupled to the elongate translating member, wherein the actuator is selectively movable relative to the body to actuate the elongate translating member between the retracted and extended positions; and (e) a force measuring feature operatively coupled with the actuator, wherein the force measuring feature is configured to detect and measure an axial force exerted on the elongate translating member by the actuator when the elongate translating member moves between the retracted and extended positions.

### Example 2

The apparatus of Example 1, wherein the elongate translating member comprises one of a guidewire or a dilation catheter.

### Example 3

The apparatus of any of the preceding Examples, wherein the force measuring feature is further configured to communicate a characteristic of the detected axial force to at least one of a user or a controller.

### Example 4

The apparatus of Example 3, wherein the characteristic comprises a magnitude of the detected axial force.

### Example 5

The apparatus of any of the preceding Examples, wherein the force measuring feature is configured to detect and measure proximally directed axial force and distally directed axial force exerted on the elongate translating member by the actuator.

### Example 6

The apparatus of any of the preceding Examples, wherein the force measuring feature comprises a transducer, wherein the transducer is configured to generate a signal in response to detecting the axial force, wherein the signal corresponds to a magnitude of the detected axial force.

### Example 7

A system comprising: (a) the apparatus of Example 6; and (b) a controller operatively coupled with the transducer, wherein the controller is configured to receive the signal from the transducer, wherein in response to receiving the signal the controller is configured to communicate the magnitude of the detected axial force to a user.

### Example 8

The apparatus of any one or more of Examples 1 through 5, wherein the force measuring feature comprises a mechanical indicator mechanism having a resilient member, wherein the resilient member is configured to deflect in response to exertion of the axial force on the elongate translating member by the actuator.

### Example 9

The apparatus of Example 8, wherein a first portion of the resilient member is coupled with the actuator and a second portion of the resilient member is coupled with the elongate translating member, wherein the resilient member is configured to transmit axial force from the actuator to the elongate translating member.

### Example 10

The apparatus of any one or more of Examples 8 through 9, wherein the mechanical indicator mechanism is configured to indicate a magnitude of the axial force exerted on the elongate translating member.

### Example 11

The apparatus of any of Example 10, wherein the mechanical indicator mechanism is configured such that the indicated magnitude is proportional to an amount of deflection of the resilient member.

### Example 12

The apparatus of any one or more of Examples 8 through 11, wherein the mechanical indicator mechanism includes a scale element having visual indicia, wherein the visual indicia is representative of axial force magnitude.

### Example 13

The apparatus of any of the preceding Examples, further comprising a light emitting element configured to emit light at a distal end of the elongate translating member.

### Example 14

The apparatus of Example 13, wherein the light emitting element comprises an illumination fiber.

### Example 15

A system comprising: (a) the apparatus of any one or more of Examples 13 and 14; (b) an energy source operatively coupled with the apparatus, wherein the energy source is configured to energize the light emitting element to emit light; (c) a light detector operatively coupled with the apparatus, wherein the light detector is configured to measure an intensity of light reflected proximally through the apparatus; and (d) a controller operatively coupled with the light detector, wherein the controller is configured to compare the measured light intensity with a predetermined light intensity to thereby identify a structural condition of the elongate translating member.

### Example 16

A method of monitoring force exerted on a translating member of a surgical instrument by an actuator, wherein the translating member comprises one of a guidewire or a dilation catheter, the method comprising: (a) exerting with the actuator an axial force on a force measuring feature of the surgical instrument; (b) transmitting the exerted axial force from the force measuring feature to the translating member, thereby causing the translating member to translate along a longitudinal axis of the surgical instrument; (c) measuring with the force measuring feature a magnitude of the exerted axial force; and (d) providing an indication of the measured magnitude to a user.

### Example 17

The method of Example 16, wherein the force measuring feature comprises a transducer, wherein the method further comprises transmitting a signal from the transducer to a controller, wherein the signal corresponds to the measured magnitude.

### Example 18

The method of Example 16, wherein the force measuring feature includes a resilient member, wherein transmitting the exerted axial force from the force measuring feature to the translating member includes deflecting the resilient member.

### Example 19

A method of monitoring the structural state of a guidewire having an illumination fiber extending longitudinally through the guidewire, the method comprising: (a) transmitting light distally through a distal end of the illumination fiber and onto a surface; (b) receiving through the distal end of the illumination fiber light reflected by the surface; (c) directing the reflected light proximally through the illumination fiber to a light detector; (d) measuring with the light detector an intensity of the reflected light; (e) comparing with a controller the measured light intensity with a predetermined light intensity; and (f) when the measured light intensity is less than the predetermined light intensity, providing an indication to a user.

### Example 20

The method of Example 19, wherein the predetermined light intensity corresponds to an intensity of light reflected proximally through the illumination fiber when the guidewire is in a non-buckled state.

### V. MISCELLANEOUS

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a body;
(b) a guide member coupled to and extending distally from the body, wherein the guide member defines a longitudinal axis;
(c) an elongate translating member operatively coupled with the body and slidably disposed relative to the guide member, wherein the elongate translating member is configured to translate relative to the body between a retracted position and an extended position for accessing an anatomical passageway;
(d) an actuator coupled to the elongate translating member, wherein the actuator is selectively movable relative to the body to actuate the elongate translating member between the retracted and extended positions; and
(e) a force measuring feature operatively coupled with the actuator, wherein the force measuring feature is configured to detect and measure an axial force exerted on the elongate translating member by the actuator when the elongate translating member moves between the retracted and extended positions.

2. The apparatus of claim 1, wherein the elongate translating member comprises one of a guidewire or a dilation catheter.

3. The apparatus of claim 1, wherein the force measuring feature is further configured to communicate a characteristic of the detected axial force to at least one of a user or a controller.

4. The apparatus of claim 3, wherein the characteristic comprises a magnitude of the detected axial force.

5. The apparatus of claim 1, wherein the force measuring feature is configured to detect and measure proximally directed axial force and distally directed axial force exerted on the elongate translating member by the actuator.

6. The apparatus of claim 1, wherein the force measuring feature comprises a transducer, wherein the transducer is configured to generate a signal in response to detecting the axial force, wherein the signal corresponds to a magnitude of the detected axial force.

7. A system comprising:
(a) the apparatus of claim 6; and
(b) a controller operatively coupled with the transducer, wherein the controller is configured to receive the signal from the transducer, wherein in response to receiving the signal the controller is configured to communicate the magnitude of the detected axial force to a user.

8. The apparatus of claim 1, wherein the force measuring feature comprises a mechanical indicator mechanism having a resilient member, wherein the resilient member is configured to deflect in response to exertion of the axial force on the elongate translating member by the actuator.

9. The apparatus of claim 8, wherein a first portion of the resilient member is coupled with the actuator and a second portion of the resilient member is coupled with the elongate translating member, wherein the resilient member is configured to transmit axial force from the actuator to the elongate translating member.

10. The apparatus of claim 8, wherein the mechanical indicator mechanism is configured to indicate a magnitude of the axial force exerted on the elongate translating member.

11. The apparatus of claim 10, wherein the mechanical indicator mechanism is configured such that the indicated magnitude is proportional to an amount of deflection of the resilient member.

12. The apparatus of claim 8, wherein the mechanical indicator mechanism includes a scale element having visual indicia, wherein the visual indicia is representative of axial force magnitude.

13. The apparatus of claim 1, further comprising a light emitting element configured to emit light at a distal end of the elongate translating member.

14. The apparatus of claim 13, wherein the light emitting element comprises an illumination fiber.

15. A system comprising:
(a) the apparatus of claim 13;
(b) an energy source operatively coupled with the apparatus, wherein the energy source is configured to energize the light emitting element to emit light;
(c) a light detector operatively coupled with the apparatus, wherein the light detector is configured to measure an intensity of light reflected proximally through the apparatus; and
(d) a controller operatively coupled with the light detector, wherein the controller is configured to compare the measured light intensity with a predetermined light intensity to thereby identify a structural condition of the elongate translating member.

16. A method of monitoring force exerted on a translating member of a surgical instrument by an actuator, wherein the translating member comprises one of a guidewire or a dilation catheter, the method comprising:
(a) exerting with the actuator an axial force on a force measuring feature of the surgical instrument;
(b) transmitting the exerted axial force from the force measuring feature to the translating member, thereby causing the translating member to translate along a longitudinal axis of the surgical instrument;
(c) measuring with the force measuring feature a magnitude of the exerted axial force; and
(d) providing an indication of the measured magnitude to a user.

17. The method of claim 16, wherein the force measuring feature comprises a transducer, wherein the method further comprises transmitting a signal from the transducer to a controller, wherein the signal corresponds to the measured magnitude.

18. The method of claim 16, wherein the force measuring feature includes a resilient member, wherein transmitting the exerted axial force from the force measuring feature to the translating member includes deflecting the resilient member.

19. A method of monitoring the structural state of a guidewire having an illumination fiber extending longitudinally through the guidewire, the method comprising:
(a) transmitting light distally through a distal end of the illumination fiber and onto a surface;
(b) receiving through the distal end of the illumination fiber light reflected by the surface;
(c) directing the reflected light proximally through the illumination fiber to a light detector;
(d) measuring with the light detector an intensity of the reflected light;
(e) comparing with a controller the measured light intensity with a predetermined light intensity; and
(f) when the measured light intensity is less than the predetermined light intensity, providing an indication to a user.

20. The method of claim 19, wherein the predetermined light intensity corresponds to an intensity of light reflected proximally through the illumination fiber when the guidewire is in a non-buckled state.
